(19) United States
(12) Patent Application Publication
(10) Pub. No.: US 2015/0295562 A1
(43) Pub. Date: Oct. 15, 2015

(54) Low Power Wireless Sensor System with Ring Oscillator And Sensors for Use in Monitoring of Physiological Data

(71) Applicants: Mangilal Agarwal, Indianapolis, IN (US); Ali Daneshkhah, Indianapolis, IN (US); Hosseign Jafarian, Indianapolis, IN (US); Sudhir Shrestha, Indianapolis, IN (US); Khodadad Varahramyan, Indianapolis, IN (US); Anthony Faiola, Indianapolis, IN (US)

(72) Inventors: Mangilal Agarwal, Indianapolis, IN (US); Ali Daneshkhah, Indianapolis, IN (US); Hosseign Jafarian, Indianapolis, IN (US); Sudhir Shrestha, Indianapolis, IN (US); Khodadad Varahramyan, Indianapolis, IN (US); Anthony Faiola, Indianapolis, IN (US)

(21) Appl. No.: 14/685,298

(22) Filed: Apr. 13, 2015

Related U.S. Application Data
(60) Provisional application No. 61/978,567, filed on Apr. 11, 2014.
(60) Provisional application No. 62/008,127, filed on Jun. 5, 2014.
(60) Provisional application No. 61/978,490, filed on Apr. 11, 2014.

(51) Int. Cl. H03K 3/012; G01N 33/00; G01N 27/22; A61B 5/00; H03K 3/03; H03K 3/011; G01N 27/414; A61B 5/08; G01N 33/497; G01N 27/04

(52) U.S. Cl. CPC H03K 3/012; G01N 33/497; G01N 33/0047; G01N 27/22; G01N 27/04; H03K 3/0315; H03K 3/011; G01N 27/4146; A61B 5/082; A61B 5/7475; A61B 5/7282; A61B 5/0004; A61B 5/743; A61B 5/746; H03K 2005/00071

(57) ABSTRACT

Wireless sensor system that integrate sensors, wireless communication module, and user interface units are disclosed. The system can include sensors fabricated for identifying hypoglycemia in the breath of a patient. The system can provide a low-power and small form factor wireless sensor system that integrates multiple sensors (e.g. resistor and capacitor based) and includes an on-chip temperature sensor in an ASIC. The disclosed system collects information from the sensors and wirelessly transmits the processed information to end user interface units, such as smart phones. The systems can be used in healthcare applications, including un-interrupted involuntary continuous monitoring of vital parameters of human body or environment, and other applications, and can be particularly adapted to monitoring hypoglycemia.

### CROSS-REFERENCE TO RELATED APPLICATIONS

This application claims the benefit of U.S. Provisional patent application Ser. No. 61/978,567 filed on Apr. 11, 2014 and entitled “Low Power Wireless Sensor System with Ring Oscillator” and claims the benefit of U.S. Provisional patent application Ser. No. 62/008,127 filed on Jun. 5, 2014, and claims the benefit of U.S. Provisional patent application Ser. No. 61/978,490, filed Apr. 11, 2014, each of which are hereby incorporated herein by reference in their entireties.

### BACKGROUND

Wireless sensors and miniature electronics are important in monitoring the vital parameters of the human body and the surrounding environment. These devices are particularly important to the elderly, children, pregnant women and the disabled. Wireless sensor systems can and do provide lifesaving assistance, particularly to these categories of patients.

Recent advances in nanoelectronics and semiconductor technology have spurred rapid progress in the development of and integration of nanosensors into application specific integrated circuit (ASIC) devices for biomedical, chemical, and other sensor applications Nanotechnology has been applied, for example, in integrated RF-powered contact lenses, and smart lens technology capable of sensing glucose level from tears. It is desirable to further adapt nanoelectronic systems to other types of biosensors.

There is, for example, a global epidemic in diabetes with a rising incident rate for both type 2 and type 1 diabetes T1D. One in ten healthcare dollars in the U.S. is spent on costs directly attributable to diabetes, with over half of these costs directly or indirectly resulting from poor maintenance of blood glucose (BG) levels. Persons with T1D require very tight monitoring of BG levels to avoid complications from not only hyperglycemia but also hypoglycemia (HYPO). An electronic sensor that monitors the volatile organic compounds corresponding to changes in human breath, therefore, is desirable for detecting HYPO.

Successful implementation of these sensor devices, however, requires low-power readout systems with robust and reliable output that is independent of environmental conditions such as temperature to operate effectively and over a reasonable time frame. Available general purpose analog to digital converters (ADC), however, consume a relatively large amount of energy that limits the lifetime of wireless sensors. Energy efficient, small foot-print, high sensitivity ring oscillator-based sensor read-out systems, particularly complementary metal-oxide-semiconductor (CMOS) technology, have been proposed to meet this need. Traditional ring oscillators, however, are temperature-dependent, and have a limited tuning frequency range. These deficiencies have been major impediments on the path to the realization of low-power sensing systems. The present disclosure addresses these and other issues.

### SUMMARY

The present disclosure describes a low power miniaturized wireless sensor system equipped with multiple sensors to monitor vital parameters and communicate the information with gateway device for healthcare and other applications. The system employs a ring oscillator design that provides a reduction in power consumption, a wide tuning frequency range, and temperature stable operation for advanced miniature sensing systems.

The present disclosure provides an application specific integrated circuit (ASIC) consisting of analog and digital sub-systems forming a system on chip (SOC) using complementary metal-oxide-semiconductor (CMOS) technology. A low power and wide tuning range current-starved-ring-oscillator design drives capacitance and resistance-based sensors using an arrangement of delay elements with two levels of control voltages. A bias unit provides these two levels of control voltages and consist of CMOS cascade current mirror to maximize voltage swing which give the oscillator wider tuning range and lower temperature induced variations.

The ASIC design uses an efficient method of analog to digital conversion and a novel sequential sensor monitoring for power management. The conversion of analog sensor input to digital is achieved by counting the number of pulses of a sensor-driver in one clock cycle.

A novel method of power management and sequential monitoring of several sensors with CMOS technology is also disclosed. A power efficient digital subsystem design includes a system management unit (SMU) that enables or disables a sensor id. The disclosed design captures the pulse waves from a sensor for a portion of clock cycles, 3 clocks out of a 16-clock cycle, for example, and transmits the signal to a counter module. As a result, the analog sub-system is at ‘on-state’ for only 3/16th fraction (18%) of the time, leading to reduced power consumption.

The system also includes a ring oscillator based temperature sensor supported with symmetrical load that detects temperature from −50° C. to 100° C. with resolution of 0.1° C.

In one embodiment of the invention, a wireless sensor system is provided comprising a sensor for producing pulse signals corresponding to a monitored biometric parameter, a ring oscillator for driving the sensor, a counter for counting the pulses produced by the sensor, a shift register for converting the pulses acquired by the counter to a serial data package, and a system management unit, the system management unit in communication with each of an output of the sensor, an input to drive the ring oscillator, a start and a stop control of the counter, and a start and a stop control of the shift register, the system management unit programmed to drive the ring oscillator to cause the sensor to produce the pulse signal, enable the counter to count pulses corresponding to the sensor data, and to start and stop the shift register to produce the serial data package, wherein the serial data package is adapted to be transmitted wirelessly through an RF transmitter.

In another aspect, a ring oscillator for use in a sensor-driver is provided. The ring oscillator includes a plurality of odd stages, each comprising a current-starved inverter, and a plurality of even stages, each comprising an inverter connected in parallel with the current starved inverters. A capacitive element connected between subsequent odd and even stages of current starved inverters and inverters and adapted to selectively produce a delay, wherein the delay through the ring oscillator is controlled by adjusting the current applied through the CMOS capacitors.

In another embodiment, a sensor device for evaluating hypoglycemia based on the breath of a patient is disclosed. The sensor device comprises a plurality of sensors forming an array, each of the sensors selected to identify a volatile organic compound (VOCs) corresponding to a component of human breath indicative of hypoglycemia, a microcontroller in communication with the plurality of sensors, a user interface in communication with the microcontroller, and a memory in communication with the microcontroller. The microcontroller is programmed to receive an input signal from the user interface indicating a request to breathe into the device to evaluate hypoglycemia, activate the sensor array to detect VOCs corresponding to a users breath, evaluate output of the array to determine a level of hypoglycemia, and store the evaluated level of hypoglycemia in memory.

The sensor device as recited can include a wireless communications system for wirelessly transmitting the evaluated level of hypoglycemia, or a display that can display the level of hypoglycemia. The sensor device can also include an alert system for providing an alert that indicates hypoglycemia has been detected.

The sensors in the sensor device can be field effect transistors, including a channel comprising a sensor material. The sensors in the sensor device can also be resistive or capacitive sensors having a sensor material comprising a nanomaterial selected from the group consisting of gold nanoparticles, carbon nanotubes, graphene, fullerene, carbon black, and combinations thereof. The nanomaterials can be coated with a surface coating or one or more functional groups selected from the group consisting of C₁-C₉ thiol-alkanes, C₁₀-C₂₀ thiol-alkanes, C₂-C₉ thiol-aromatics, C₁₀-C₂₀ thiol-aromatics, and combinations thereof to detect ketones, aldehydes, alcohols, and nitrates; can comprise a material selected from the group consisting of polypyrrole, low-density polyethylene (LDPE), poly(ethylene-block-ethylene oxide) (PE-b-PEO), polyethylene glycol (PEG), poly methyl methacrylate (PMMA), poly(vinylidene fluoride-hexafluoropropylene) (PVDF-HFP), and combinations thereoffor acetone detection; or can comprise aromatic or aliphatic surface coatings for detection of aromatic and aliphatic carbon compounds.

These and other aspects of the invention will become apparent from the following description. In the description, reference is made to the accompanying drawings which form a part hereof, and in which there is shown a preferred embodiment of the invention. Such embodiment does not necessarily represent the full scope of the invention and reference is made therefore, to the claims herein for interpreting the scope of the invention.

### BRIEF DESCRIPTION OF THE DRAWINGS

FIG. 1 is a block diagram of a sensor system and corresponding wireless communication system constructed in accordance with one embodiment of the present disclosure.

FIG. 2 is a block diagram of the sensor system of FIG. 1 illustrating an analog and a digital subsystem.

FIG. 3 is a simplified block diagram of the analog subsystem of FIG. 2.

FIG. 4 is a block diagram of the analog subsystem of FIG. 2 illustrating a ring oscillator.

FIG. 5 is a block diagram of the digital subsystem of FIG. 2.

FIG. 6 is a circuit diagram of a current starved inverter for use in a ring oscillator of the type shown in FIG. 4.

FIG. 7 is a circuit diagram of a ring oscillator circuit of the type shown as a block in FIG. 4.

FIG. 8 is a circuit diagram of a cascaded control voltage in a symmetrical load current starved inverter.

FIG. 9 is a circuit diagram of a dummy transistor.

FIG. 10 is a circuit diagram of a delay stage constructed with the dummy transistor of FIG. 9.

FIG. 11 is a circuit diagram of a ring oscillator useful in the system of FIG. 2.

FIG. 12 is a plot of output frequency of the ring oscillator as the control voltage is varied.

FIG. 13 is a plot of percentage change in output frequency of the ring oscillator with variations in ambient temperature.

FIG. 14 is a simplified schematic of a field effect transistor including a sensing material selected for detecting volatile organic compounds (VOC's) in the breath of a human.

FIG. 15 is a block diagram of a sensor device incorporating sensors of the type shown in FIG. 14 and in communication with a wireless communication system.

FIG. 16 is a perspective view of a sensor device incorporating elements of FIG. 16 shown mounted in a housing.

FIG. 17 is a flow chart illustrating the steps for evaluating breath samples using a sensor device as shown in FIGS. 15 and 16 to evaluate disease states and particularly hypoglycemia.

### DETAILED DESCRIPTION

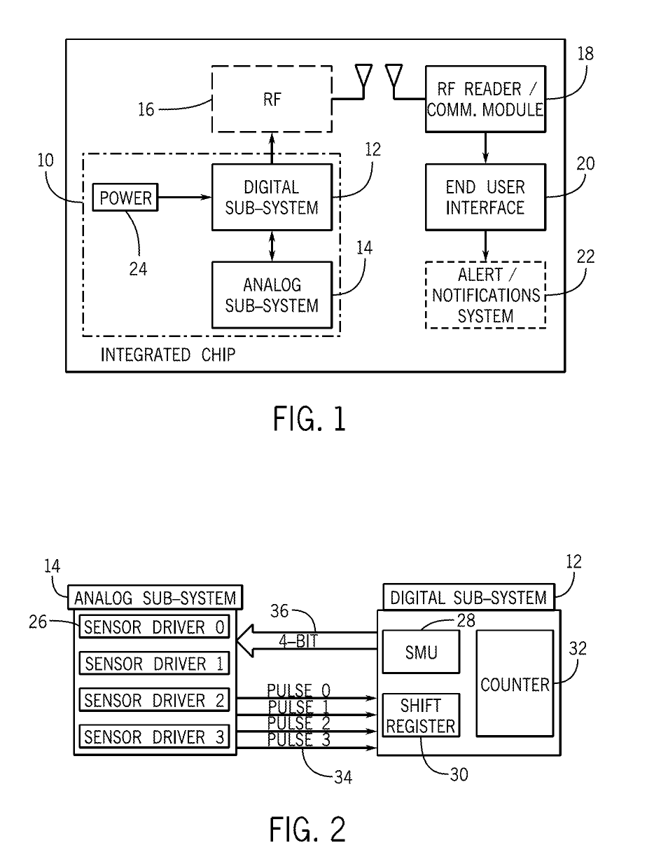

Referring now to FIG. 1, a system diagram illustrating a sensor system 10 for monitoring biometric data and providing RF communications of the data and constructed in accordance with the present disclosure is shown. The sensor system 10 includes a digital sub-system 12 and an analog sub-system 14, and is preferably constructed in a system on chip (SOC) or application specific integrated circuit (ASIC) format. As shown here, the digital sub-system 12 can be in communication with an RF transmitter or transceiver 16, which can communicate sensor data to an external RF reader 18. The reader 18 can include, as shown here, an end user interface 20 and alert/notification system 22. A power supply 24 provides power to the digital and analog sub-systems.

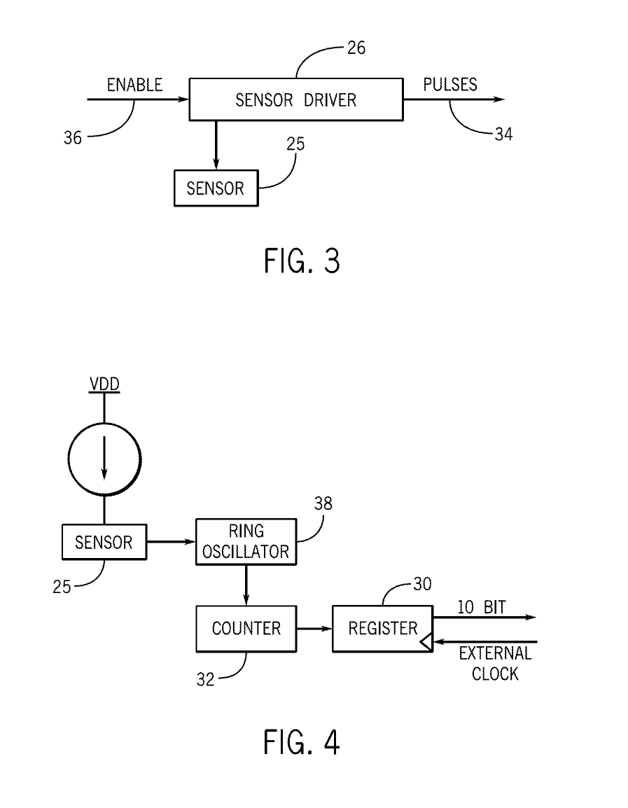

Referring now also to FIGS. 2 and 3, a block diagram of the sensor system 10 and a corresponding block diagram of the analog subsystem are shown. The analog subsystem 14 comprises a plurality of sensors 25 and corresponding sensor drivers 26, which provide pulse signals 34 to the digital subsystem 12. The digital subsystem 12 includes a shift register 30, which receives the pulse signals 34, and a corresponding counter 32. A system management unit (SMU) 28 is provided in the digital system 12, and the SMU 28 provides enable signals 36 to enable the sensor drivers 26.

In operation, the sensors 26 in the analog subsystem 14 generates pulse waves 34. The output sensor voltage is amplified, and converted to digital pulses 34. The pulses output from the analog subsystem 14 are directed to the digital subsystem 12. The digital subsystem 12 converts the analog sensor output to digital by counting the number of pulses produced by a sensor-driver with the counter 32. The SMU 28 enables or disables the sensors 26 in the analog system 14, and controls sensor drivers to capture sensor output voltage. The counter 32 and corresponding shift register 30 convert output from the sensors 26 from parallel to serial data which can be transmitted via the RF transmitter 16 (FIG. 1). The transmitted data can be biometrical data such as, but not limited to, heart rate, blood pressure, ECG, oxygen saturation, EEG, and temperature data. Referring again to FIG. 1, this data can be transmitted to a reader 18 which can be, for example, a computer, cell phone, smart phone, etc. By integrating biosensors, the system-on-chip can detect and monitor vital parameters of the human body and transmits the detected data to an end user interface (e.g. smart phone).

Referring still to FIGS. 2 and 3, during operation, the analog voltage variation detected by the sensors 25 is converted to pulse waves 34 frequencies in the analog sub-system 12. The width of pulse wave 34 depends on the voltage generated by sensor 25. Increasing input voltage when the width of the pulse wave 34 decreases results in higher frequency output. The captured signal is amplified within the analog sub-system 12 and transmitted to the digital sub system 14. Although the number can be varied, as shown here, the analog subsystem 12 can include four sensor drivers 26, and corresponding sensors 25.

Referring still to FIG. 2, the sensor drivers 26 work based on frequency change with input voltage or current, which in turn is dependent on sensor parameters such as resistance or capacitance. Referring now also to FIG. 4, the core of the sensor-drivers can be a ring oscillator 38. Here, the output analog signal from a sensor 25 is fed into the ring oscillator 38. The output frequency of the ring oscillator 38, which in turn is fed to the counter 32, is modulated by the sensor output. The counter 32 measures the frequency of the signal and produces digital output, which can be, for example, 8 or 10 bits. This output is fed to the shift register 30. The digital output of the counter 32 is dependent on the frequency of the ring oscillator 38. The ring oscillator 38 therefore effectively converts the analog signal from the sensor 25 to the digital form without the need for a traditional analog to digital convertor ADC, thus reducing the power consumption and the size of the system. A suitable ring oscillator 38 is described below with reference to FIGS. 6-11.

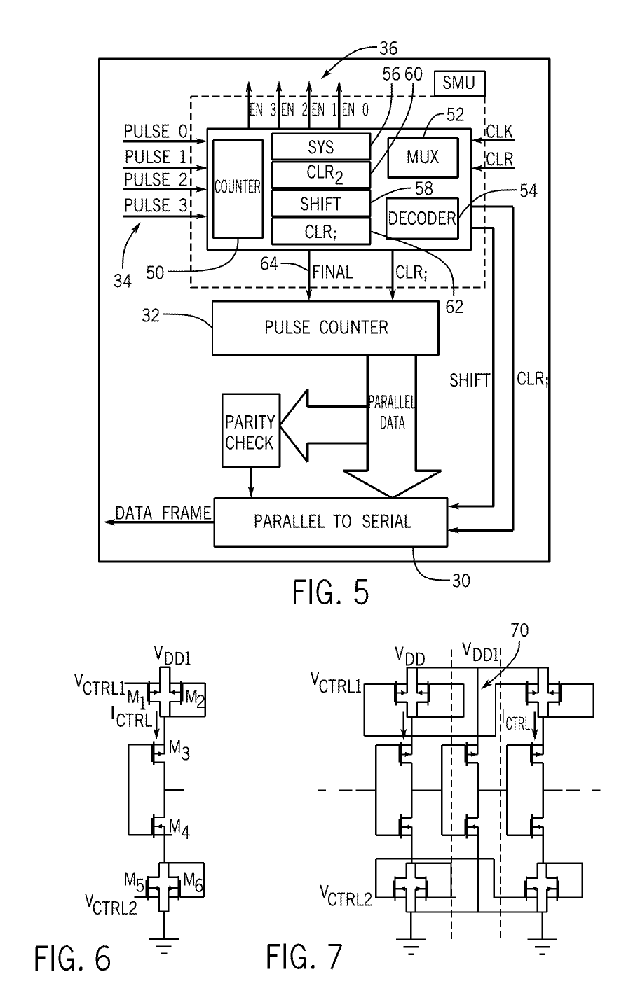

Referring again to FIGS. 1 and 2 and also to FIG. 5, as described above, the received pulse signals 34 from the sensor 25 in the analog sub-system 14 are processed and interfaced to the digital sub-system 16. According to the basic strategy of the system, the digital sub-system 12 sequentially calls each sensor-driver 26 to receive the pulse wave data generated from the corresponding sensors 25 within the analog sub-system 12. After receiving pulses, the number of pulses is counted per clock cycle. And in the final stage, the counted number of pulses is packed in a package of data for serial transmission. After data packing, the sensor 25 is forced to become inactive by digital sub-system.

Referring again to FIG. 5, as shown here, in one embodiment, the digital subsystem 12 includes a SMU 28 that includes an on-board counter 50, multiplexer 52, and a decoder 54. The SMU 28 produces enable signals 36 (En 0,1,2,3) for sequentially enabling each of the sensors 25 as described above, and receives pulse data at pulse inputs 34 corresponding to each of the sensors 25. The SMU 28 produces four internal control signals, system signal (SYS) 56, Shift 54, and clear signals clear signal (CLR2) 60 and (CLR3) 62. The Shift 58 and CLR3 signals start and stop the shift register 30, respectively, for converting parallel data produced by the counter 32 to serial. The Sys 56 and CLR2 60 signals change state each sixteen-clock period. These two signals produce the enable outputs 36 for driving the sensors 25 in the analog sub-system 14 and encode the generated pulses in the sensor-drivers 26 as output of the SMU 28.

In operation the on-board counter 50 counts clock pulses to produce system control signals. The decoder 54 generates the enable signal, and the multiplexer 52 produces the pulse signal to be provided to the pulse counter 32 though output 64. Although other configurations are possible, here the on-board counter 50 is a four bit counter for counting clock signals, while the pulse counter 32 is an 8 bit counter.

To generate the enabling signal and process the final signal, the multiplexer 52 and decoder 54 sample the CLR2 60 and SYS 56 level signals. During the three clocks including CLR3 falling, between CLR3 falling and SYS rising, and SYS rising, the decoder 54 generates the enable signal 36 to activate a sensor driver 26 in the analog subsystem. The multiplexer 52 transmits the pulse signal to the pulse counter 32 along the “final” output line 64. Each sensor 25 is therefore monitored for 3 clocks during 64 clocks. During the clock cycle, the counter 32 counts the number of pulses in the received signal 64 to produce eight bit parallel data output that shows the number of the carried pulses in one clock, and the counter keeps the outputs until next zero in CLR3. After pulse counting is complete, the SMU 28 sends the shift 58 signal to the shift register 30 to begin conversion of the parallel data acquired by the pulse counter 32 to serial data. The SMU 28 produces the CLR3 signal to stop the conversion and data packing During conversion, the shift register 30 produces a data package produced of 11 bits. Therefore, 11 clocks after the zero state in the Shift signal 58, the CLR3 signal 62 fall to zero from high logic level. The time interval between counting finishing and resetting is 14 clocks. Therefore, during the time interval, the parallel data is shifted and outputted in serial form.

After completing data packing, the SMU 28 sends an enable signal 36 for next sensor-driver 26 and repeats all process again for a new sensor-driver 26. System Management Unit (SMU) 28 manages all processes and allocates the power supply for the operating modules rather than all modules in the system to minimize power consumption. Low power consumption is achieved by activating a sensor 25 for only a fraction of the clock cycle. Here a sensor 25 is activated only during 3 clocks of 16 clocks cycles while all other sensors remain inactive. The result is that each sensor is activated once every 64-clocks (4×16). Therefore the analog sub-system 14 consumes the power for one sensor during 3 clocks of 16 clocks cycle. With this method, the power consumption is reduced by 82% compared to a configuration where all the sensors are continuously activated. The sensors sampling rate depends on the clock of the system. As a result a higher sampling rate can be achieved by using higher frequency.

Referring again to FIG. 4 and also to FIGS. 6-11, a ring oscillator 38 constructed in accordance with the present disclosure is shown. The oscillator 38 comprises a temperature-stable, low-power ring oscillator with a wide tuning frequency range, suitable for implementation in an ASIC. The oscillator 38 includes a chain of delay elements consisting of a current-starved inverter and a CMOS capacitor, which can further delay the system. The delay is controlled by changing the current through the delay elements. The presented design is applicable in advanced sensing systems, including biomedical, chemical, and other sensors.

A conventional ring oscillator consists of an odd number of inverters (N) connected in series that form a closed loop path. The frequency of oscillation is determined by the overall delay in the inverter loop, which in turn is dependent on the delay in each inverter. The delay in an inverter is controlled by the current through the transistors that make up the inverter (called as control current, I_{CTRL}). In this model, an increase in the current reduces the delay. If V_{OSC} is the amplitude of oscillating output signal, the dependence of the delay in each inverter is given by:

$$τ = \frac{{V}_{OSC} \times {C}_{G}}{{I}_{CTRL}} \qquad ( 1 )$$

Where C_{G} is the sum of gate-source parasitic capacitances of the MOSFETs. The MOSFET parasitic capacitance further depends on the width and length of the gate of the transistor. The frequency of oscillation is given by:

$${f}_{OSC} = \frac{1}{2 N \times τ} \qquad ( 2 )$$

Combining the above equations, we get the frequency of oscillation as a function of the control current as shown below:

$${f}_{OSC} = \frac{{I}_{CTRL}}{2 N \times {V}_{OSC} \times {C}_{G}} \qquad ( 3 )$$

A ring oscillator 38 constructed in accordance with one embodiment of the present disclosure is shown in FIG. 6. In the ring oscillator of FIG. 6, the conventional current starved inverter with power switching is replaced by current starved inverter with symmetrical load. The symmetrical load generates higher current, increasing the sensitivity of the ring oscillator and providing higher frequency of oscillation. To further improve stability of the oscillator, a simple inverter 70 can be placed between two current starved inverters with symmetrical load as shown in FIG. 7.

In reference to FIG. 6, the source to gate voltage for M₁ and drain to source voltage for M₅ can be written as

V_{SG}_{_{M1}}=(V_{DD}−V_{CTRL1}) and V_{GS}_{_{M5}}=V_{CTRL2} (4)

Thus, the drain currents for these two transistors can be written in term of control voltages as:

$$\begin{matrix}{I}_{{SD}_{M 1}} = \frac{{μ}_{{pC}_{OX}}}{2} \frac{{W}_{M 1}}{{L}_{M 1}} {( {V}_{DD} - {V}_{CTRL 1} + {V}_{T} )}^{2} & ( 5 ) \\ {I}_{{SD}_{M 5}} = \frac{{μ}_{{NC}_{OX}}}{2} \frac{{W}_{M 5}}{{L}_{M 5}} {( {V}_{CTRL 2} - {V}_{T} )}^{2} & ( 6 )\end{matrix}$$

Where μₚ and μₙ are average hole and electron mobility in the channel, C_{OX} is Oxide Capacitance, W is gate width, L is gate length, and V_{T} is threshold voltage, respectively. Equations (5) and (6) show that the drain current varies as the square of the control voltage, I_{SDM1} decreases with V_{CTRL1} and I_{DSM5} increases with V_{CTRL2}. As symmetrical load builds up more current, it increases these effects. From FIG. 6, the control current (I_{CTRL}) can be written as the sum of source to drain current of M₁ and M₂,

I_{CTRL}=I_{SD}_{_{M1}}+I_{SD}_{_{M2}}, and (7)

V_{SG}_{_{M2}}=V_{SD}_{_{M1}}. (8)

That leads to

$${I}_{CTRL} = {I}_{{SD}_{M 1}} + \frac{{μ}_{{nC}_{ox}}}{2} \frac{{W}_{M 2}}{{L}_{M 2}} {( {V}_{{SD}_{M 1}} + {V}_{T} )}^{2} \qquad ( 9 )$$

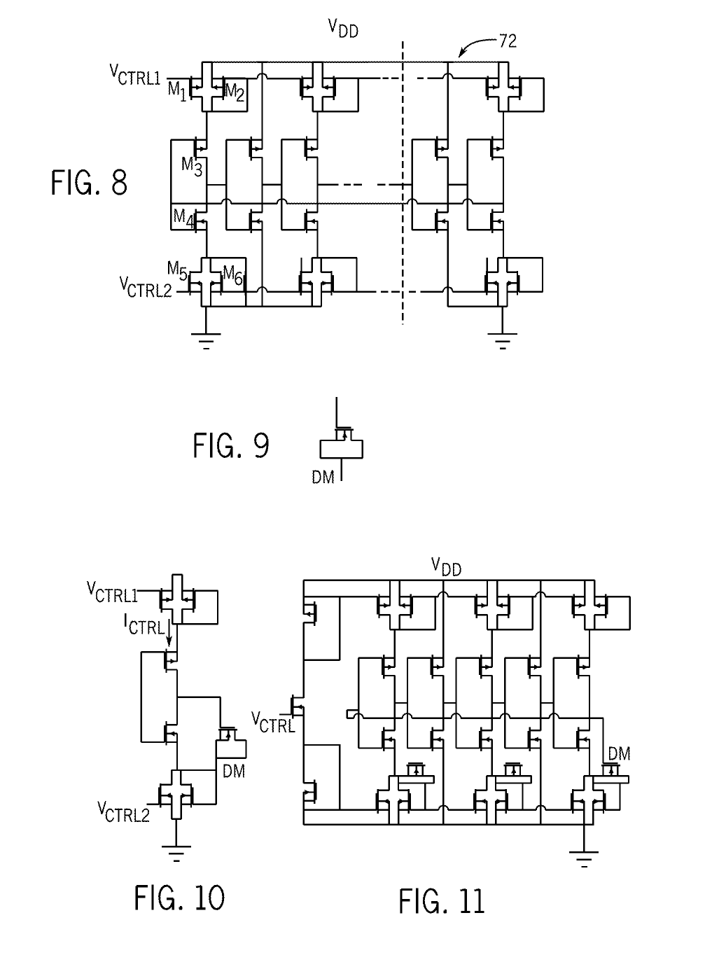

MOSFET M₂ is always in saturation region, and increase in V_{CTRL1} increases V_{SDM1}, which leads to a nonlinear increase in the current at M₂. It eventually increases the total generated current entering the MOSFETs M₃ and M₄. To further enhance the stability, except for the first stage, direct connections to control voltage for all current starved elements are removed. This was achieved by connecting the symmetrical load 72 to its preceding stage as shown in FIG. 8.

Delay in an inverter constructed with CMOS technology is in the range of several picoseconds. To reduce the number of inverters in the oscillator, and also to increase the system stability, dummy transistors are used as capacitors, which increase the delay at each stage. Dummy transistor (DM), shown in FIG. 9, provides delay control through the control voltage. The variable capacitance (dummy transistor) is implemented after each current starved element, depicted in FIG. 10. Thus, the ring oscillators 38 shown here consists of two delay dummy transistor units, and a current starved inverter with symmetrical load as shown in FIG. 11. A dummy transistor placed between the inverter and voltage controlled delay elements also provides better temperature stability. The ring oscillator includes 5 delays stages as shown in FIG. 11. Three of the delay units are voltage controlled elements, where the first stage is controlled by input voltage (V_{CTRL}) which in turn generates control voltage for the following two stages.

As described above with reference to FIG. 4, the input voltage to the ring oscillator 38 is provided by the output a sensor 25. The output of the ring oscillator is directed to the counter 32 in the digital system 16, where it can be processed as described above. Other stages in the processing may also be provided as discussed above.

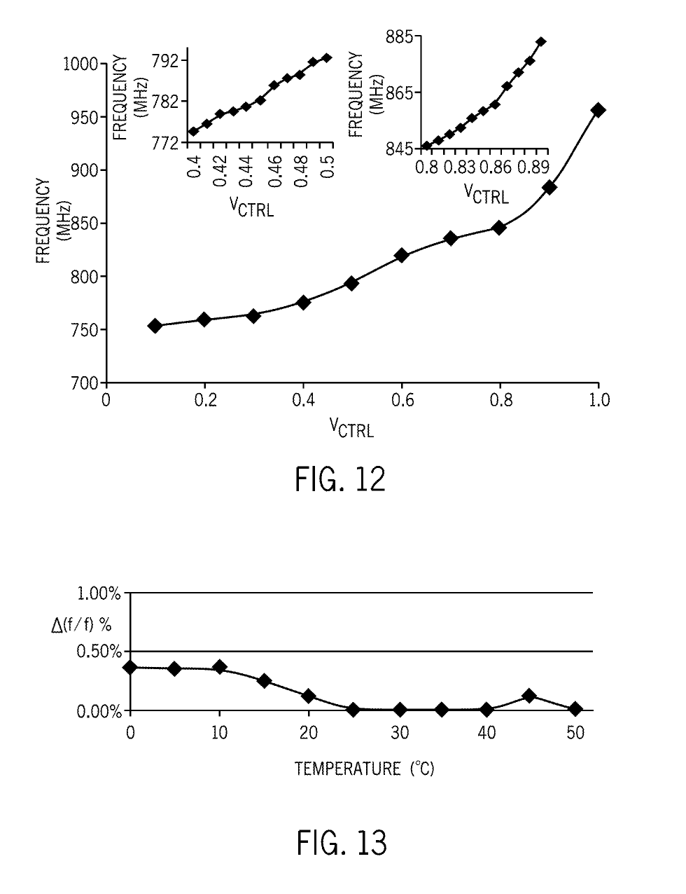

In one embodiment of the invention, the oscillator design described above shown in FIG. 11 was constructed using 180 nm CMOS technology and a 1.8 V power supply. Output frequency with respect to the applied control voltage, change in the output frequency with variation in ambient temperature, and overall power consumptions were analyzed. A plot of output frequency of the oscillator when the control voltage was varied from 0.1 to 1 V is shown in FIG. 12. For the given range of control voltages, the output frequency varies from 753 MHz to 956 MHz, a change of 203 MHz. This corresponds to a change of 22.55 MHz in output frequency for each 100 mV change in the control voltage; an increased response compared to previously reported low-power oscillator designs. The insets of FIG. 12 show the results for 0.4 to 0.5 V and 0.8 to 0.9 V ranges. These figures depict linear sub-regions within the shown control voltage range. The curve has a higher slope within 0.8 to 0.9 V range. Percentage change in output frequency of the design with variations in ambient temperature is shown in FIG. 13. The control voltage for this simulation was set to 0.8 V. It is observed that the change in the ring oscillator output frequency is less than 0.5% when the ambient temperature varies from 0 to 50 degrees C. The result shows more stable operation of the oscillator against ambient temperature variations. The power consumption of the presented design was measured to be 1.2 nW, although additional losses due to parasitic effects and leakage currents may be present in some fabricated chips, and power consumption may also be higher in some fabricated chips. The low power consumption along with higher frequency response and temperature stability makes the presented design a better candidate for low-power sensor system applications.

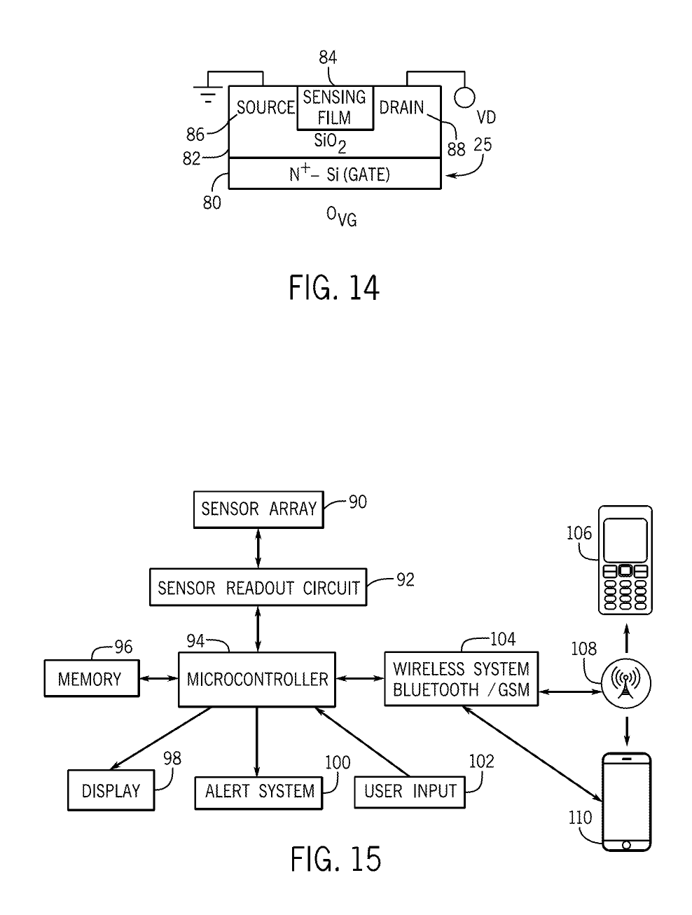

Referring now to FIG. 14, a sensor 25 that can be used in a sensor system 10 of the type described above, and which can be particularly configured for use in monitoring the breath of a patient to identify disease states, is shown. As shown here, the sensor 25 can be a field effect transistor (FET) based sensor. A doped silicon substrate 80 with oxide and gold layers are used. Silicon constitutes the gate of the device, gold is patterned for source 86 and drain 88 using photolithography techniques, oxide constitute the dielectric, and the sensing material 84 disposed between the gold source and drain electrodes 86 and 88 constitutes the channel of the transistor. A plurality of sensor elements 25 can be fabricated on a sensor array. Each sensor element 25 includes two electrodes 86 and 88 and a selected sensing material 84, which can be coated in the channel between the electrodes to detect specific volatile organic compounds (VOCs) as described below.

Referring still to FIG. 14, in a sensor array (90; FIG. 15) the sensing material 84 in each sensor 25 can be selected to respond to one or more of the VOCs that can be used as biomarkers to identify disease states in human breath. Human breath is composed of inhaled air, CO2, water vapor, some concentration of proteins, and a mixture of VOCs. The VOCs are generated either as byproducts of internal metabolic reactions, as gases produced for physiological signaling roles, or as metabolites from inhaled atmospheric air, and VOCs have been used to identify specific health conditions, as described below. In a sensor array, at least one sensor element can be provided in the array to respond to each of the identified VOCs that are consistent with a selected disease state.

The sensing material 84 is selected to transform chemical concentrations of analytes in human breath to electrical signals. Analytes interact with sensor materials 84 to cause a change in the electronic or physical property of the material, resulting in a change in conductivity (change in resistance) or a change in the permittivity (change in capacitance) of the sensor material 84. The sensing material 84 can include, for example, finely tuned polymeric materials or nanoparticles coated with organic surface coatings which are sensitive to different VOC analytes. These types of sensors can therefore detect changes in the composition of VOC's in human breath that are, for example, permanently altered by disease states. Sensors have been developed and used to identify disease states such as types of cancer, including lung, breast, colorectal, prostate, and gastric, and also to identify whether a person has diabetes. (See Gang Peng, Meggie Hakim, Yoav Y. Broza, Salem Billan, Roxolyana Abdah-Bortnyak, Abraham Kuten, Ulrike Tisch, Hossam Haick, Detection of lung, breast, colorectal, and prostate cancers from exhaled breath using a single array of nanosensors, British Journal of Cancer 2010, 103, 542-551 Jae Kwak, Michelle Gallagher, Mehmet Hakan Ozdener, Charles J. Wysocki, Brett R. Goldsmith, Amaka Isamah, Adam Faranda, Steven S Fakharzadeh, Meenhard Herlyn, A. T. Johnson, Volatile biomarkers from human melanoma cells, Journal of Chromatography B 2013; 40 J. Hofbauer, H. Dressel, J. Seissler, A. R. Koczulla, D. Nowak, R. A. Jones, Analyse der Ausatemluft mittels Elektronischer Nase bei Patienten mit Diabetes mellitus, Pneumologie 64, V266.) Each of the references cited herein are incorporated by reference for their description of sensor devices detecting VOC's. Descriptions of sensors for detecting VOC's in breath based sensing are also disclosed in “VOLATILE ORGANIC COMPOUND SENSORS, AND METHODS OF MAKING AND USING THE SAME,” attorney docket number 144578.00127, filed on even day herewith, which is also hereby incorporated by reference in its entirety.

Similar systems can be used to detect the transitory changes that can occur in, for example, hypoglycemia (HYPO). Here, the sensors 25 are again constructed with sensing materials 84 to detect transient disease states from VOCs that are present in human breath, as well as significant changes in the relative levels of VOCs. VOCs indicative of HYPO have been identified based on analysis of the breath of individuals identified as experiencing HYPO by, for example, diabetes alert dogs (DADs) using gas chromatograph/mass spectrometry (GC/MS) data. Table I identifies VOCs that have been shown experimentally to correlate with hypoglecemia, in the approximate ranges shown:

TABLE I
VOC's having correlation with hypoglycemia: (ppb—parts per billion)

| | Methyl | Pentyl | | | | | Ethyl | |
|---|---|---|---|---|---|---|---|---|
| Acetone | nitrate | Nitrate | Ethanol | Methanol | Propanol | Methane | benzene | Isoprene |
| 1.2-1,880 ppb | 1-1,000 ppb | 1-2,000 ppb | 13-1,000 ppb | 160-2,000 ppb | 1.6-170 Ppb | 10-170 Ppb | 12-580 ppb | 1-2,000 ppb |

Additional data concerning the concentration of VOC's in breath can be collected, for example, by correlating blood glucose level data with breath samples collected when diabetic patients experience HYPO and normoglycemia. The samples can be collected, for example, in Tedlar bags, and transferred from the Tedlar reusable bags to deactivated glass vials with sorbent materials. Subsequently, samples are transferred to a solid phase microextracion (SPME) matrix at 80° C. The SPME matrix is inserted into the GC/MS instrumentation (Agilent GC7890A (GC) and 5975C (MS)) directly. The elements of the VOC signature panel are run through the GC/MS to confirm the identity of analytes previously determined solely by their mass spec signature, and to determine specific analyte concentrations for the HYPO signature breath profile. The number of components found may, in some cases, be increased by utilizing SPME fibers with different matrices including polydimethylsiloxane, carboxen, and divinylbenzene coatings.

The GC/MS data can be analyzed using an Automated Mass Spectral Deconvolution and Identification System, and the results subjected to principal component analysis (PCA) and other multivariate statistics to determine the HYPO signature breath profile from specific changes in the concentration of VOCs. Distinct VOC signatures for Normal breath and HYPO breath can be produced and used in sensor fabrication to create sensor arrays for identifying HYPO.

VOC concentrations such as those shown in Table 1 and collected as described above can be used to construct a sensor array to include individual sensors 25 having sensing materials 84 selected to identify VOCs associated with hypoglycemia, thereby providing a readout of whether breath analyzed with the sensor exhibits characteristics consistent with hypoglycemia. Although a number of materials can be used as sensing material 84, one suitable material is gold nanoparticles coated with dodecanethiol, available from OceanNanoTech LLC, Springdale, Arkansas. Similar materials can be synthesized as described in “Diagnosing lung cancer in exhaled breath using gold nanoparticles,” Nat. Nanotechnol., vol. 4, no. 10, pp. 669-673, October 2009, G. Peng, U. Tisch, O. Adams, M. Hakim, N. Shehada, Y. Y. Broza, S. Billan, R. Abdah-Bortnyak, A. Kuten, and H. Haick. In alternate embodiments, the nanomaterials can be selected from the group consisting of gold nanoparticles, carbon nanotubes, graphene, fullerene, carbon black, and combinations thereof.

These nanomaterials are coated with a surface coating or one or more functional groups selected from the group consisting of C₁-C₉ thiol-alkanes, C₁₀-C₂₀ thiol-alkanes, C₂-C₉ thiol-aromatics, C₁₀-C₂₀ thiol-aromatics, and combinations thereofusing a ligand exchange method in order to be suitable to detect ketones, aldehydes, alcohols, and nitrates (See M. Badea, A. Amine, G. Palleschi, D. Moscone, G. Volpe, and A. Curulli, “New electrochemical sensors for detection of nitrites and nitrates,” J. Electroanal. Chem., vol. 509, no. 1, pp. 66-72, August 2001). Other coatings can include polycyclic aromatic hydrocarbons (PAH), carboxylic acid, decanethiol, dodecanethio, tert-dodecanethiol, 4-methoxy-toluenethiol, 2-nitro-4-trifluoro-methylbenzenethiol, and 2-mercaptobenzoxazole.

Additional sensing materials that can be used in these applications include polypyrrole, low-density polyethylene (LDPE), poly(ethylene-block-ethylene oxide) (PE-b-PEO), polyethylene glycol (PEG), poly methyl methacrylate (PMMA), poly(vinylidene fluoride-hexafluoropropylene) (PVDF-HFP), and combinations thereoffor acetone detection (J.-B. Yu, H.-G. Byun, M.-S. So, and J.-S. Huh, “Analysis of diabetic patient's breath with conducting polymer sensor array,” Sens. Actuators B Chem., vol. 108, no. 1-2, pp. 305-308, July 2005.) 2,3-diaminonapthalene which is sensitive to nitrates (A. K. Nussler, M. Glanemann, A. Schirmeier, L. Liu, and N. C. Nüssler, “Fluorometric measurement of nitrite/nitrate by 2,3-diaminonaphthalene,” Nat. Protoc., vol. 1, no. 5, pp. 2223-2226, December 2006.) and aromatic and aliphatic surface coatings for aromatic and aliphatic carbon compounds. (G. Peng, M. Hakim, Y. Y. Broza, S. Billan, R. Abdah-Bortnyak, A. Kuten, U. Tisch, and H. Haick, “Detection of lung, breast, colorectal, and prostate cancers from exhaled breath using a single array of nanosensors,” Br. J. Cancer, vol. 103, no. 4, pp. 542-551, August 2010.)

The sensors can be FET sensors, as described above, resistive, or capacitive sensors. To fabricate the sensors in a resistor or capacitor model, the sensing material is 84 placed between two conducting electrodes 86, 88 and change in resistance or capacitance when breath samples are exposed is measured. Fabrication techniques can include photolithography, self-assembly, spin-casting, drop-casting, spray-coating, chemical-bath-deposition, hot-pressing, evaporation, and sputtering. The layer-by-layer (LbL) self-assembly technique can be advantageously used to with a wide choice of materials and a precise control of film properties at the molecular level to create thin, sensitive channels of sensing material 84.

To test operation, fabricated sensor devices including sensing material 84 are placed in an air-tight chamber. Individual VOCs (with appropriate concentrations) and breath samples are passed through the chamber. Resistance or capacitance can be measured using a Keithley semiconductor characterization instrument. The temperature, pressure, and humidity of the chamber are also measured.

As the VOCs are injected into the chamber, and come in contact with the sensing material 84, they are adsorbed on the material. The attachment of the target analyte molecule to the sensing material 84, through reversible chemical bonding, intermolecular interactions, or physical adsorption will alter the electronic or physical properties of the sensing material 84. For a transistor-based device, the change in electronic property of the channel film can be measured through the drain current and the gate and drain voltages. The relationship between the saturation region drain current and gate voltage of a FET is shown in equation (1).

$${I}_{D} = \frac{μ {C}_{O} W}{2 L} {( {V}_{G} - {V}_{T} )}^{2} \qquad ( 1 )$$

Where, ID=drain current, μ, =mobility, C0=gate capacitance, W and L=width and length of channel, VG=gate voltage, and VT=turn on voltage.

The drain current in the sub-threshold region is given by equation (2).

$${I}_{D} = \frac{μ {C}_{O} W}{L} ( {V}_{G} - {V}_{T} - \frac{{V}_{D}}{2} ) {V}_{D} \qquad ( 2 )$$

Where, VD=drain voltage. As the analytes attach to the sensing material 84, the change in drain current and threshold voltage will be measured. The layer-by-layer (LbL) self-assembly can be used to enable the deposition of films as thin as a few molecules thick, which enables the construction of thinner channel layers and consequently more sensitive FETs to changes caused by adsorbed VOCs. Although FETs are shown and described, other electronic devices can be constructed as sensors. For example, diode models can be constructed to include sensing materials where adsorption of the target analyte causes changes in charge mobility.

Referring now to FIG. 15, a block diagram of a portable sensor system 91 that can incorporate a sensor array 90 comprising sensors 25 of the type described above is shown. The sensor system 91 comprises a microcontroller 94, corresponding memory 96, display 98, and user interface 102. The microcontroller 94 is in communication with a sensor readout circuit 92 which, in turn, drives the sensor array 90. The microcontroller 94 is also in communication with an alert system 100 and wireless communications system 100.

In operation, the individual current levels from the sensor array 90 can be collected by readout circuit 92 and analyzed by microcontroller 94 for the corresponding breath status, as described more fully below. The sensor readout circuit 92 manages the sequence of reading the sensor array 90, and input the acquired data to the microcontroller 94, and performing sensor resetting or clearing functions after the read-out or when it receives such an instruction from the microcontroller 94. The microcontroller 94 process the data using a set of instructions provided to compute a breath status value, and can save a date and time stamp in memory 96, where the data can be used as a comparator to evaluate changes in the breath. The breath status value can also be displayed on a local display 98, and wirelessly transmitted the wireless communications system 104 to the patient, designated caregivers, or both. The microcontroller 94 can also activate the alert system 100, which can provide audio or visual signals to a user. The alert system 100 can include, for example, light emitting diodes (LEDs), circuitry for producing audio alerts, and other types of devices.

The wireless communications system 104 can include various types of wireless communications devices, including, for example, a radiofrequency, Bluetooth, or GSM communication system that is in communication with the microcontroller 94, and one or more corresponding antenna 108. Although individual blocks are shown in the block diagram illustrated to represent circuitry having specific functions, it will be apparent that circuitry for performing functions shown in the blocks can be constructed using one or more electronic component, and that blocks can be separated and combined. For example, the wireless system 104 may consist of more than one component, or more than one integrated circuit (IC) chip. Further, various blocks can be combined into a single IC chip or other device.

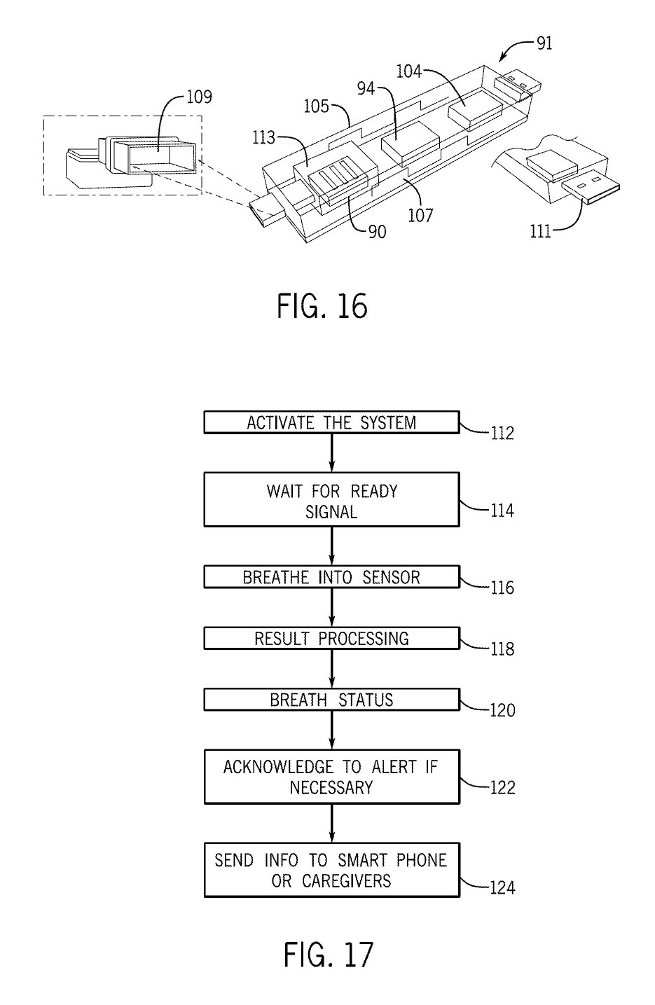

Referring now to FIG. 16, an exemplary embodiment of a portable sensor device system 91 is shown illustrating components located within a housing 105. As shown here, in one embodiment of the invention, the sensor array 90, microcontroller 94, and wireless system 104 can be placed on a printed circuit board (PCB) 107 and interconnected through etched copper lines. Antennas 108 and a rechargeable battery can also be provided in the housing 105.

Referring still to FIG. 16, a breath inlet 109 and USB connector 111 are provided in the housing 105, and can be located at opposite ends of the housing 105 as shown. In use, a user breathes into the breath inlet 108, and the breath of the user passes through a filter (not shown0 and enters a chamber 113 that encloses the sensor array 90. The filter prevents dust, smoke, and bigger air-borne particulates from entering the air chamber 113. The air chamber 113 consists of a number of compartments to divide the incoming breath into several streams with each stream focused to one sensor 25 in the array 90. Each flow stream can narrow as it approaches the corresponding sensor 25 to increase the flow rate when the air contacts the sensor 25. The breath exits the air chamber 113 and is released out through air flow vents positioned along the two side-walls of the system housing (not shown). One or more antenna, such as a microstrip antenna, can be provided in the housing 105, and can advantageously be mounted to the two inner sidewalls of the device housing 105. A rechargeable battery can be used to power the circuitry in the sensing device 91, and can be positioned on the bottom of the housing 105, below the PCB 109. The USB 111 can be used to recharge the battery, although other methods of recharging a battery will be apparent to those of ordinary skill in the art. The USB 111 can, in some applications, also be used as a user interface. The USB 111 could, for example, be used to access and retrieve data stored in memory 96.

Referring now also to FIG. 17, a flow chart illustrating operation of sensor device 91 is shown. A test for hypoglycemia begins with the user activating the sensor device 91 (step 112). The user can activate the device, for example, by activating a pushbutton or other indicator associated with a user input device (102; FIG. 15). After the sensor device 91 is activated, the user waits until a ready signal is received (step 114). When the sensor array 90 and corresponding microcontroller 94 are ready to analyze the breath of the user, a ready signal can be provided. The ready signal can, for example, be a message provided on a display 98 corresponding to the sensor device 91, a signal transmitted to a cell phone, or activation of a dedicated LED or sound alarm generated by appropriate circuitry associated with the alert system 100 in communication with microcontroller 94. After the ready signal is active, the user can breathe into the sensor array 90 (Step 116). As described above, this step can involve breathing into an inlet 109 with corresponding filter device.

Referring still to FIG. 17, after the breath sample is received on the sensor array 90, the microcontroller 94 can activate a “read” of the data by, for example, activating a sensor readout circuit 92. The microcontroller 94 can then acquire data from the sensor array 90, and process the data (step 118). After analysis is complete, the microcontroller 94 determines a breath status (step 120) and then can write or transmit the results, which can be, for example NORMAL, PROBABLE HYPO or HYPO, or can include numerical data. In some applications, for example, the microcontroller 94 can write the results to a dedicated display 98 (FIG. 15).

Alternatively, the results can be transmitted to a cell phone 106, 110. In some applications, the results can be both locally displayed on display 98 and wirelessly transmitted to a smart phone, cell phone, computer, or other type of personal computing device. For example, the user can observe the result on a local sensor display 98, acknowledge the result if a HYPO breath status is indicated (step 122), and optionally activate a pushbutton or other device associated with user interface 102 to transmit the information to an external device of the type described above. (step 124) The transmitted data can be transmitted to the user and/or the user's selected caregiver. The communication between the sensor device 91 and other wireless devices will be encrypted to ensure patient privacy. Each sensor device 91 will be encrypted with a PGP key that can be selected by the user at the time of device calibration, and must be similarly programmed into the application corresponding to the receiving cell phone, smart phone, or other communications device. The PGP key will be transmitted by the sensor device 91 along with the raw sensor data.

Referring again to FIGS. 15 and 17, as described above, a smart phone, cell phone, or other communication device 106, 110 can be in communication with the sensor device 91. The smart phone, cell phone, or other computing device can be programmed to include an application that serves as the interface for observing and analyzing results history, as well as initial setup of the sensor device 91. In operation, therefore, the sensor system 91 can collect, process, and interpret sensor data, and store that data in memory 96. The data can then be accessed through the user interface 102 or through the wireless system 104.

The smart phone application can also download primary and secondary caregiver phone numbers and setup information to the sensor device 91 through wireless communications system 104. The patient and caregivers' smart phones can then receive and integrate breath status data into an ongoing historical log, and the log data can be provided, for example, on a data visualization dashboard. The smart phone interface can display, for example, patient name, date and time of most recent breath status readings, the current breath status, with the corresponding color code for that status, and the time-line breath status data visualization.

The smart phone application can also include menus. The menus can include, for example, a “Home” icon for accessing the other menu functions; a “Sensor” menu to access the setup tools for downloading caregiver phone numbers into the sensor device 91 and to perform sensor calibration; a “Security” menu for preventing unauthorized access to all transmitted personal health data, such as GSM encrypted algorithms including secure encryption protocols for blocking invaders through its peer-to-peer authentication; a “Dashboard” menu that provides an interface for viewing past and current readings of breath status; a “Social” menu enabling access to tools for sending or receiving messages between the patient and the caregiver; a “Profile” menu enabling patients to input their personal information, name, age, etc.; and “Setup” menu enabling access to general system configuration.

After the patient blows into the device, a breath status alert will be sent automatically to the electronic devices identified in memory 96. The patient and the caregiver will be able to review test results, which can be, for example, identified with words such as “NORMAL,” “PROBABLE HYPO,” or “HYPO”. The status can also be color-coded, and provided with a date and time stamp. In some applications, the display can also provide access to historical data. For example, the user can be provided with a function to scroll left and right to see current and past readings, allowing comparison of levels throughout the day. Alerts can be evaluated by the patient in coordination with their caregiver to apply corrective measures to bring the status level back to Normal.

It should be understood that the methods and apparatuses described above are only exemplary and do not limit the scope of the invention, and that various modifications could be made by those skilled in the art that would fall within the scope of the invention. For example, although specific analog and digital configurations are shown, it will be apparent that components of the systems can be combined or structured in different formats. Programmable devices can also be used to provide similar functions. The sensors described herein can be used with various monitoring applications in addition to medical applications. Although specific hardware elements are described, it will be apparent to those of ordinary skill in the art that equivalent elements can be used, and that the construction can be re-configured to reduce the number of components in the system. To apprise the public of the scope of this invention, the following claims are made:

We claim:

1. A wireless sensor system comprising:
a sensor for producing pulse signals corresponding to a monitored biometric parameter;
a ring oscillator for driving the sensor;
a counter for counting the pulses produced by the sensor;
a shift register for converting the pulses acquired by the counter to a serial data package; and
a system management unit, the system management unit in communication with each of an output of the sensor, an input to drive the ring oscillator, a start and a stop control of the counter, and a start and a stop control of the shift register, the system management unit programmed to drive the ring oscillator to cause the sensor to produce the pulse signal, enable the counter to count pulses corresponding to the sensor data, and to start and stop the shift register to produce the serial data package, wherein the serial data package is adapted to be transmitted wirelessly through an RF transmitter.

2. The wireless sensor system of claim 1, wherein the ring oscillator comprises a first stage including a current starved inverter and a second stage comprising a symmetrical load.

3. The wireless sensor system of claim 1, wherein the ring oscillator comprises first stage comprising a current starved inverter, a second stage comprising a simple inverter, a third stage comprising a current starved inverter, and a fourth stage comprising a symmetrical load.

4. The wireless sensor system of claim 1, wherein the ring oscillator comprises a first stage comprising a current starved inverter, and a variable capacitance is placed between the inverter and a subsequent inverter.

5. The wireless sensor system of claim 4, wherein the variable capacitance comprises a dummy transistor.

6. The wireless sensor system of claim 1, further comprising a plurality of sensors, and where the system management unit is further programmed to sequentially activate the plurality of sensors for a portion of clocks in a clock cycle selected to enable acquisition of a sensor pulse output, and to deactivate the activated sensor for the remainder of the clock cycle, wherein power is conserved.

7. The wireless sensor system of claim 1, further comprising an RF transmitter adapted to receive the data package and to transmit the data package to a wireless communications device.

8. A ring oscillator for use in a sensor-driver, comprising:
a plurality of odd stages, each comprising a current-starved inverter;
a plurality of even stages, each comprising an inverter connected in parallel with the current starved inverters; and
a capacitive element connected between subsequent odd and even stages of current starved inverters and inverters and adapted to selectively produce a delay, wherein the delay through the ring oscillator is controlled by adjusting the current applied through the CMOS capacitors.

9. The ring oscillator of claim 8, wherein the capacitive element comprises a dummy transistor.

10. The ring oscillator of claim 8, comprising three odd stages and two even stages.

11. A sensor device for evaluating hypoglycemia based on the breath of a patient, the device comprising:
a plurality of sensors forming an array, each of the sensors comprising a sensor material selected to identify a volatile organic compound (VOCs) corresponding to a component of human breath indicative of hypoglycemia;
a microcontroller in communication with the plurality of sensors;
a user interface in communication with the microcontroller;
a memory in communication with the microcontroller, wherein the microcontroller is programmed to:
receive an input signal from the user interface indicating a request to breathe into the device to evaluate hypoglycemia;
activating the sensor array to detect VOCs corresponding to a users breath;
evaluating the array to determine a level of hypoglycemia; and
storing the evaluated level of hypoglycemia in memory.

12. The sensor device as recited in claim 11, further comprising a wireless communications system for wirelessly transmitting the evaluated level of hypoglycemia.

13. The sensor device as recited in claim 11, further comprising a display, wherein the processor is further programmed to write an evaluation of the level of hypoglycemia on the display.

14. The sensor device as recited in claim 11, further comprising an alert system for providing an alert that indicates hypoglycemia has been detected.

15. The sensor device as recited in claim 11, wherein the sensors are field effect transistors.

16. The sensor device as recited in claim 15, wherein the sensor material comprises a channel in the field effect transistor.

17. The sensor device as recited in claim 15, wherein the sensor is a resistive or capacitive sensor and the sensor material comprises a nanomaterial selected from the group consisting of gold nanoparticles, carbon nanotubes, graphene, fullerene, carbon black, and combinations thereof.

18. The sensor device as recited in claim 17, wherein the nanomaterial is coated with a surface coating or one or more functional groups selected from the group consisting of C₁-C₉ thiol-alkanes, C₁₀-C₂₀ thiol-alkanes, C₂-C₉ thiol-aromatics, C₁₀-C₂₀ thiol-aromatics, and combinations thereof.

19. The sensor device as recited in claim 15, wherein the sensor is a resistive or capacitive sensor and the sensor material comprises a material selected from the group consisting of polypyrrole, low-density polyethylene (LDPE), poly(ethylene-block-ethylene oxide) (PE-b-PEO), polyethylene glycol (PEG), poly methyl methacrylate (PMMA), poly(vinylidene fluoride-hexafluoropropylene) (PVDF-HFP), and combinations thereof.

20. The sensor device as recited in claim 15, wherein the sensor material comprises aromatic or aliphatic surface coatings.
